(19) United States
(12) Patent Application Publication
(10) Pub. No.: US 2020/0003759 A1
(43) Pub. Date: Jan. 2, 2020

(54) METHODS FOR IDENTIFYING METAP-2 MODULATORS

(71) Applicant: Zafgen, Inc., Boston, MA (US)

(72) Inventors: James E. Vath, Lynnfield, MA (US); Bryan Burkey, Ipswich, MA (US)

(21) Appl. No.: 16/484,603

(22) PCT Filed: Feb. 12, 2018
(86) PCT No.: PCT/US2018/017805

§ 371 Date: Aug. 8, 2019

Related U.S. Application Data
(60) Provisional application No. 62/457,355, filed on Feb. 10, 2017.
(60) Provisional application No. 62/457,347, filed on Feb. 10, 2017.

(51) Int. Cl. G01N 33/50

(52) U.S. Cl. CPC G01N 33/5064

(57) ABSTRACT

Disclosed herein, in part, are methods for identifying MetAP-2 inhibitors to address the treatment of obesity and related diseases as well as other ailments favorably responsive to MetAP-2 modulator treatment.

### CROSS REFERENCE TO RELATED APPLICATIONS

This application is a national stage filing under 35 U.S.C. § 371 of PCT/US2018/017805, filed Feb. 12, 2018, which claims the benefit of, and priority to, U.S. Provisional Patent Application No. 62/457,347, filed Feb. 10, 2017, and U.S. Provisional Patent Application No. 62/457,355, filed Feb. 10, 2017, the contents of each of which are hereby incorporated by reference in their entirety.

### BACKGROUND

MetAP-2 encodes a protein that functions at least in part by enzymatically removing the amino terminal methionine residue from certain newly translated proteins such as glyceraldehyde-3-phosphate dehydrogenase (Warder et al. (2008) J. Proteome Res. 7:4807). Increased expression of the MetAP-2 gene has been historically associated with various forms of cancer. Molecules inhibiting the enzymatic activity of MetAP-2 have been identified and have been explored for their utility in the treatment of various tumor types (Wang et al. (2003) Cancer Res. 63:7861) and infectious diseases such as microsporidiosis, leishmaniasis, and malaria (Zhang et al. (2002) J. Biomed. Sci. 9:34). Notably, inhibition of MetAP-2 activity in obese and obese-diabetic animals leads to a reduction in body weight in part by increasing the oxidation of fat and in part by reducing the consumption of food (Rupnick et al. (2002) Proc. Natl. Acad. Sci. USA 99:10730).

Such MetAP-2 inhibitors may be useful as well for patients with excess adiposity and conditions related to adiposity including type 2 diabetes, hepatic steatosis, and cardiovascular disease (via e.g. ameliorating insulin resistance, reducing hepatic lipid content, and reducing cardiac workload). For example, over 1.1 billion people worldwide are reported to be overweight.

The MetAP2 inhibitor beloranib has produced consistent and clinically meaningful weight loss in clinical trials of patients with obesity, type 2 diabetes, Prader-Willi syndrome (PWS), and hypothalamic injury-associated obesity. In patients with type 2 diabetes, beloranib produced 13% weight loss and a 2.0% reduction in HbA1c over 26 weeks of treatment. Beloranib was generally well tolerated in preclinical testing. However, in clinical trials of beloranib in patients with obesity, PWS, or type 2 diabetes, adverse events (AEs) of venous thromboembolism occurred in beloranib-treated patients despite being otherwise generally well-tolerated. These AEs included superficial thrombophlebitis, deep vein thrombosis, and pulmonary embolism (PE), including two fatal PEs in patients with PWS that resulted in cessation of beloranib development.

While there has been significant effort to find viable MetAP-2 modulating compounds, it remains a challenge to find MetAP-2 inhibitors with reduced side effects, including reduced thromboembolic effects, and the success rate of such compounds remains poor and unchanged. However, MetAP-2 inhibitors may be useful in the treatment of non-oncologic disorders including metabolic diseases, obesity and or a co-morbidity thereof, type 2 diabetes, latent autoimmune diabetes, chronic inflammatory disease and impaired wound healing, and accordingly, methods for identifying MetAP-2 inhibitors are clearly needed to address the treatment of non-oncologic disorders as well as other ailments favorably responsive to MetAP-2 modulator treatment.

### SUMMARY

The present disclosure provides, at least in part, methods for identifying MetAP-2 modulators, e.g., inhibitors. Such methods may comprise exposing cells or tissue to a MetAP-2 inhibitor candidate compound; measuring the inhibition of proliferation in the cell or tissue in discrete time intervals; and selecting the candidate compound as suitable for treatment by identifying whether the candidate compound shows minimal inhibition of cell proliferation at a set time exposure to the cell or tissue.

Also provided herein are methods for identifying a candidate MetAP-2 inhibitor compound suitable for treatment of a human disorder, comprising: exposing a cell to a potential MetAP-2 candidate compound in a culture medium; retrieving a sample from the cell and/or culture medium at one or more predetermined time points; analyzing the sample for increased or decreased expression levels of at least one gene each selected from the group consisting of p53, p21, eNOS, PAI-1, TM, RF, KLF2, MDM2, and vimentin; and identifying the compound as suitable for treatment of obesity based on the increased expression level or decreased expression level.

Also provided herein is a method for identifying MetAP-2 inhibitors having minimal persistant cell proliferation and therefore suitable for human treatment of disorders, comprising: exposing test cells or tissue to a MetAP-2 inhibitor candidate compound for a first incubation time; performing a washout of the candidate compound from the test cells or tissue after the first incubation time; continuing incubation of the cells in the absence of the candidate compound for a second incubation time; measuring the inhibition of proliferation in the test cells or tissue in discrete time intervals; and selecting the candidate compound as suitable for treatment by identifying whether the candidate compound shows minimal inhibition of cell proliferation at a designated time after the washout to the cells or tissue.

Further provided herein are other methods for identifying a candidate MetAP-2 inhibitor compound suitable for treatment of disorders, including for example, a method comprising exposing a cell to a potential compound in a culture medium; retrieving a sample from the cell and/or culture medium at one or more predetermined time points; analyzing the sample for increased or decreased levels of PAI-1 cell protein; and identifying the compound as suitable for treatment based on the increased expression level or decreased PAI-1 cell protein levels.

### BRIEF DESCRIPTION OF THE DRAWINGS

FIG. 1A depicts results of inhibition of EC proliferation assessed in HUVECs (n=5 replicates/group); and after incubation in various concentrations of drug for 72 hours.

FIG. 1B depicts results of inhibition of EC proliferation assessed in HUVECs after incubation with Compound A (n=4-9 replicates/group) for 1, 4, 8, 24, 48, and 72 hours. Inhibition of proliferation was measured at 1, 4, and 72 hours for 6 and 12 nM concentrations of compound A.

FIG. 1C depicts inhibition of EC proliferation assessed in HUVECs after incubation with beloranib (n=4-8 replicates/group) for 1, 4, 8, 24, 48, and 72 hours. Inhibition of proliferation was measured at 1, 4, and 72 hours for 6 and 12 nM concentrations beloranib.

FIG. 2A depicts p21 protein levels after exposure to Compound A (left) and beloranib (right) at discrete time intervals and concentrations. (Students t-test vs 0 nM control; n=5 to 10 replicates per test condition pooled from 3 experiments; * P<0.05; ** P<0.005)

FIG. 2B depicts TM protein levels after exposure to Compound A (left) and beloranib (right) at discrete time intervals and concentrations. (Students t-test vs 0 nM control; n=5 to 10 replicates per test condition pooled from 3 experiments; * P<0.05; ** P<0.005)

FIG. 2C depicts PA1 protein levels after exposure to Compound A (left) and beloranib (right) at discrete time intervals and concentrations. (Students t-test vs 0 nM control; n=5 to 10 replicates per test condition pooled from 3 experiments; * P<0.05; ** P<0.005)

FIG. 3 depicts measurements of vWF levels after exposure to compounds at discrete time intervals and concentrations.

FIG. 4 depicts measurements of p53 protein levels after exposure to compounds at discrete time intervals and concentrations.

FIG. 5 depicts the results of Compound A, beloranib, or vehicle administered s.c. Q3D to dogs (n=6/group). Data are median, 25th and 75th percentile, and min/max value. Antithrombin III: LLN=130%, ULN=175%. Platelet count: thrombocytopenia threshold for dogs ≤50,000 cells/μL, LLN=161,000 cells/μL, ULN=466,000 cells/μL. ULN and LLN based on ±2 standard deviations from the mean. Two-way ANOVA analysis with Tukey's multiple comparison for difference from vehicle by day of the study, * p<0.05, ** p<0.01, *** p<0.001.

FIG. 6A depicts an overview of a HUVEC study design. HUVECs (n=9 replicates/group) were incubated with drug or vehicle (control) for 1 hour, cells were washed, and incubated an additional hour with drug or vehicle and again washed and immediately harvested or placed in drug free media and harvested at 24, 48, or 72 hours. Drug concentration was 10 nM.

FIG. 6B shows the results of cell proliferation (DNA content) assessed 72 hours after the start of the first incubation.

FIG. 6C shows levels of drug bound to MetAP2 assessed 2, 24, 48, or 72 hours after the start of the first incubation.

FIG. 6D shows THX 1-6 levels assessed 2, 24, 48, or 72 hours after the start of the first incubation.

FIG. 7A shows the results of HepG2 cells (n=3) incubated with Compound A or beloranib for 2 hours, washed, incubated in drug free media, and harvested at 2, 24, 48 or 72 hours. Cell lysates were used to measure MetAP2 protein with drug bound in the active site, normalized to total MetAP2.

FIG. 7B shows the results of HUVECs (n=3) incubated with Compound A or beloranib for 2 hours, washed, incubated in drug free media, and harvested at 2, 24, 48 or 72 hours. Cell lysates were used to measure MetAP2 protein with drug bound in the active site, normalized to total MetAP2.

FIG. 8A shows the results of HUVECs (n=3 replicates/group) incubated with Compound A or beloranib for 1 hour followed by assessment of cellular concentrations of drug.

FIG. 8B shows the results of HUVECs (n=3 replicates/group) incubated with Compound A or beloranib for 1 hour followed by assessment of drug bound to the MetAP2 active site (B).

FIG. 9 depicts inhibition of HUVEC proliferation for candidate compounds (solid lines) vs. beloranib (dashed line).

### DETAILED DESCRIPTION

Methods disclosed herein relate in part to the discovery of factors relevant to coagulation and drug safety for MetAP-2 inhibitors, including e.g., the need for providing reduced endothetial cell function.

The features and other details of the disclosure will now be more particularly described. Before further description of the present disclosure, certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### Definitions

“Treating” includes any effect, e.g., lessening, reducing, modulating, or eliminating, that results in the improvement of the condition, disease, disorder and the like.

“Individual,” “patient,” or “subject” are used interchangeably and include any animal, including mammals, preferably mice, rats, other rodents, rabbits, dogs, cats, swine, cattle, sheep, horses, or primates, and most preferably humans The pharmaceutical compositions of the present disclosure can be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like). The mammal treated in the methods of the present disclosure is desirably a mammal in which treatment of obesity or weight loss is desired. “Modulation” includes antagonism (e.g., inhibition), agonism, partial antagonism and/or partial agonism.

In the present specification, the term “therapeutically effective amount” means the amount of the subject compound that will elicit the biological or medical response of a tissue, system or animal, (e.g. mammal or human) that is being sought by the researcher, veterinarian, medical doctor or other clinician. The pharmaceutical compositions of the present disclosure are administered in therapeutically effective amounts to treat a disease. Alternatively, a therapeutically effective amount of a pharmaceutical composition is the quantity required to achieve a desired therapeutic and/or prophylactic effect, such as an amount which results in weight loss.

### Methods

The present disclosure provides, at least in part, methods for identifying MetAP-2 inhibitors suitable for treatment of a disorder in a human patient, (e.g., suitable for therapeutic use), comprising: exposing cells and/or tissue to a MetAP-2 inhibitor candidate compound; measuring the inhibition of proliferation in the cells or tissue (e.g., in discrete time intervals); and selecting the candidate compound as suitable for treatment by identifying whether the candidate compound shows minimal inhibition of cell proliferation at designated time of exposure (e.g., 1-72 hours) to the cells or tissue.

In certain embodiments, cells for use in the described methods may be e.g., endothelial cells, e.g. venous endothelial cells or RBE4 cells, or may be, for example, human umbilical vein endothelial cells (HUVEC). In other embodiments, the cells may be any type of sensitive cell such as cancer cells, e.g., hepatocellular carcinoma cells (e.g., HLE, HLF, Hep3b, HepG2), intestinal cells (e.g., Caco2), nerve cells, and/or stem cells (e.g., pluripotent stem cells).

Contemplated methods may include an exposing step that comprises exposing the cells or tissue to a MetAP-2 inhibitor candidate compound at a concentration of about 0.1 nM of about 18 nM or more, for example, at a concentration of about 0.15 nM to about 5 nM, about 0.1 nM to about 10 nM, about 1.5 nM to about 5 nM, or about 1 nM to about 18 nM or more, at a concentration of about 15 nM to about 30 nM or more, or about 1 nM to about 100 nM or more, or for example, at a concentration of about 1 nM to about 20 nM or more.

In an embodiment, exposing steps that form part of the contemplated methods may comprise exposing cells and/or tissue (e.g, human endothetial cells) to a MetAP-2 inhibitor candidate compound at a concentration of about 10 to 100 times or more, 1 to 100 times, 10 to 50 times, 15 to 50 times, about 15 to about 30 times, or about 5 to about 20 times the IC₅₀ (or, in alternative embodiment, the IC₁₀) of the MetAP-2 inhibitor candidate compound against MetAP-2 e.g., in the same type of test cell. In an explanatory embodiment, if the IC₅₀ of the candidate compound is 1 nm, a contemplated exemplary concentration is 10 nm of the candidate compound. Alternatively, a contemplated exposing step comprises exposing the cells to a MetAP-2 inhibitor candidate compound at a concentration of about 1 to about 50 times or more, about 5 to about 40 times, about 5 to about 20 times, or more, of the IC₉₀ against MetAP-2 of the candidate compound in the same type of test cell. For example, the concentration may be about 6 to about 30 times or more, or e.g., about 20 times the IC₅₀ or IC₉₀.

Also contemplated herein are methods for identifying MetAP-2 inhibitor compounds suitable for therapeutic use that include an exposing step that comprises exposing the cells to a MetAP-2 inhibitor candidate compound at a concentration of about 1 to about 50 times of more, about 5 to about 40 times or more, about 5 to about 20 times, or 10 to about 100 times or more, (e.g., about 6 or about 20 times) of the EC₅₀ (or in alternative embodiment, the EC₁₀) of the candidate compound exposed to HUVEC cells for 72 hours, e.g., the EC₅₀ measured in HUVEC cells at 72 hours. In an alternate embodiment, the exposing step comprises exposing the cells to a MetAP-2 inhibitor candidate compound at a concentration of about 1 to about 50 times or more, about 5 to about 40 times, or about 5 to about 20 times, or more, of the EC₉₀ of the candidate compound when exposed to HUVEC cells for 72 hours. For example, the exposing step includes exposing the cells to a MetAP-2 inhibitor candidate compound at a concentration of about 5 to about 25 times or more, e.g. about 6 times or about 20 times, the EC₉₀ of the candidate compound when exposed to HUVEC cells for 72 hours.

In some embodiments, the exposing step comprises exposing the cells to a MetAP-2 inhibitor candidate compound at a concentration of about 5 to about 50 times or more the EC₅₀ of the candidate compound when exposed to HUVEC cells for 72 hours.

Contemplated methods may include selecting the candidate compound as suitable for treatment by identifying whether the candidate compound shows minimal inhibition of cell proliferation at a designated time of exposure to the cells, e.g., at a designated time of 1 hour, 4 hours, 12 hours, 18 hours, 24 hours, 48 hours, or 72 hours. For example, the designated time of exposure may be 4 hours or may be 24 hours.

At the designated time of exposure (e.g., 4 or 24 hours), a minimal inhibition of cell proliferation at 4 hours may be less than 50%, less than 40%, less than 25%, less than or about 15 or 10%, or less than or about 5%, or in some embodiments, may be undetectable. Alternatively, the minimal inhibition may be less than (e.g., less than 20%) the inhibition of cell proliferation of beloranib. It is appreciated that the percentage of cell proliferation is based on a maximum inhibition in the cell

Contemplated methods may further comprise measuring p21 cell protein. For example, selecting the candidate compound may further comprise identifying whether the candidate compound increases p21 protein concentration more than about 4 fold at 72 hours exposure to the venous endothelial cells and/or identifying whether the candidate compound significantly increases p21 protein at a MetAP-2 inhibitor concentration of 10 nM or 20 nM or more at short exposure time (e.g., 1-12 hours, for example, 4 or 8 hours).

Contemplated methods may further comprise measuring thrombomodulin concentration and/or measuring PAI-1 cell protein concentration, and/or one or more of vWF, p53, D-Dimer, and vimentin protein.

For example, provided herein are methods for identifying MetAP-2 inhibitors suitable for human treatment of disorders, comprising: providing a concentration parameter of MetAP-2 selected from the group consisting of IC₅₀, IC₉₀, and EC₅₀, (or EC₁₀) as measured at 72 hours in a HUVEC cell, and EC₉₀ as measured at 72 hours in a HUVEC cell; exposing HUVEC to a MetAP-2 inhibitor candidate compound at a concentration of about 1 to about 50 times, e.g. about 5 to about 40 times (or about 5 to about 25 times, e.g., about 6 times or 20 times) the concentration parameter; measuring the inhibition of proliferation in the test cells at 4 hours or at 24 hours; and selecting the candidate compound as suitable for treatment by identifying whether the candidate compound shows less than or about 15% inhibition (or less than about 10%) of cell proliferation at about 4 hours of exposure to the HUVEC cells.

In certain embodiments, selecting the candidate compound may further comprise identifying whether the candidate compound increases p21 protein concentration more than, for example, about 4 fold at 72 hours exposure to the venous endothelial cells.

In certain embodiments, a contemplated method for identifying MetAP-2 inhibitors disclosed herein may comprise measuring, for example, thrombomodulin concentration. In certain other embodiments, a contemplated method disclosed herein may include measuring, for example, PAI-1 cell protein concentration and/or one or more of vWF, p53, vimentin protein, and/or D-dimer.

Another aspect of the present disclosure provides a method for identifying a candidate MetAP-2 inhibitor compound suitable for treatment of a disorder, comprising: exposing a cell to a potential MetAP-2 compound in a culture medium; retrieving a sample from the cell and/or culture medium at one or more predetermined time points; analyzing the sample for increased or decreased expression levels of at least one gene each selected from the group consisting of p53, p21, eNOS, PAI-1, TM, RF, KLF2, MDM2, and vimentin; and identifying the compound as suitable for treatment of a disorder based on the increased expression level or decreased expression level.

A further aspect of the present disclosure provides methods for identifying a candidate MetAP-2 inhibitor compound suitable for treatment of a disorder, comprising: exposing a cell (e.g., HUVEC cell) to a potential compound in a culture medium; optionally retrieving a sample from the cell and/or culture medium at one or more predetermined time points; analyzing the sample for increased or decreased levels of PAI-1 cell protein; and identifying the compound as suitable for treatment of obesity based on the increased expression level or decreased PAI-1 cell protein levels. Identification of increased or decreased PAI-1 protein may be useful toward identifying drugs with reduced side effects such as thrombosis characteristics.

In another aspect, a method for identifying MetAP-2 inhibitors having minimal persistant cell proliferation and therefore suitable for human treatment of disorders, e.g., suitable for human therapeutic use, is provided comprising: exposing test cells or tissue to a MetAP-2 inhibitor candidate compound for a first incubation time; performing a washout of the candidate compound from the test cells or tissue after the first incubation time (e.g., at 1, 2, 6, 8, 24, 48 or 72 hours); continuing incubating of the cells in absence of the compound for a second incubation time (e.g., an additional 12 hours, 24 hours, 48 hours, or 72 hours); measuring the inhibition of proliferation in the test cells or tissue in discrete time intervals; and selecting the candidate compound as suitable for treatment by identifying whether the candidate compound shows minimal inhibition of cell proliferation at a designated time (e.g., 24 hours, 48 hours or 72 hours) after the washout (and/or after the second incubation time) to the cells or tissue. It can be appreciated that one or more additional washout of compounds can be performed at a third incubation time (e.g., 4 hours, 24 hours), for example.

Methods provided herein may further comprise assessing efficacy of the candidate compound for the disorder in a cell, tissue, organ or animal. In certain embodiments, one or more contemplated methods may be used to select a MetAP-2 inhibitor compound to treat a disorder such as for example, a non-oncologic disorder, e.g. a metabolic disease. In certain other embodiments, the disorder may be, for example, obesity and/or a co-morbidity thereof. In further embodiments, the disorder may be, for example, chronic inflammatory disease or impaired wound healing.

In certain embodiments, the disorder may be, for example, an inflammatory disease. For example, the inflammatory disease may be selected from the group consisting of inflammatory bowel disease, Kawasaki disease, Sjogren's syndrome, systemic lupus erythematosus, rheumatoid arthritis, psoriatic arthritis, chronic obstructive pulmonary disease, and psoriasis.

Exemplary co-morbidities or other disorders include cardiac disorders, endocrine disorders, respiratory disorders, hepatic disorders, skeletal disorders, psychiatric disorders, metabolic disorders, and reproductive disorders.

Exemplary cardiac disorders include hypertension, dyslipidemia, ischemic heart disease, cardiomyopathy, cardiac infarction, stroke, venous thromboembolic disease and pulmonary hypertension. Exemplary endocrine disorders include type 2 diabetes and latent autoimmune diabetes in adults. Exemplary respiratory disorders include obesity-hypoventilation syndrome, asthma, and obstructive sleep apnea. An exemplary hepatic disorder is nonalcoholic fatty liver disease. Exemplary skeletal disorders include back pain and osteoarthritis of weight-bearing joints. Exemplary metabolic disorders include Prader-Willi Syndrome and polycystic ovary syndrome. Exemplary reproductive disorders include sexual dysfunction, erectile dysfunction, infertility, obstetric complications, and fetal abnormalities. Exemplary psychiatric disorders include weight-associated depression and anxiety.

In certain embodiments, candidate MetAP-2 inhibitors may be an irreversible inhibitor. In certain embodiments, the irreversible inhibitor may covalently bind, for example, to His231 of MetAP-2 via, e.g., a spiro epoxide moiety present on the irreversible inhibitor, upon administration. For example, a contemplated candidate MetAP-2 inhibitor may be an analog of, e.g., fumagillin. In some embodiments, candidate MetAP-2 inhibitors are compounds having an IC₅₀ against MetAP-2 of about 0.01 nM to about 50 nM, about 0.25 nM to about 5 nM, or about 0.1 nM to about 50 nM, e.g., an IC₅₀ of <0.05 μM, or about 0.001 μM to about 0.5 μM,or about 0.15 μM to about 0.5 μM. For example, a MetAP-2 candidate compound that may be used in one or more contemplated methods may have an IC₅₀ against MetAP-2 of about 0.1nM to about 5 nM.

In certain embodiments, a candidate MetAP-2 inhibitor may be represented by:

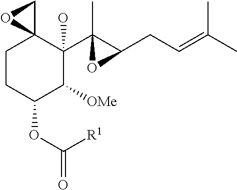

wherein R¹ may selected from C₁₋₈alkylene, C₂₋₈alkenylene, heterocyclyl, C₃₋₆cycloalkyl, —NR^{a}-C₁₋₈alkylene, —NR^{a}-C₂₋₈alkenylene, and —NR^{a}-C₃₋₆cycloalkyl; wherein R¹ may be substituted by a substituent selected from the group consisting of: carboxy, —O—C(O)—NR^{a}R^{b}, —C(O)—O—C₁₋₆alkyl, phenyl (optionally substituted by substituent selected from NR^{a}R^{b}, C₁₋₆alkoxy (optionally substituted by a substituent selected from the group consisting of NR^{a}R^{b}, C₁₋₆alkyl, and heterocyclic)), C₁₋₆alkylene (optionally substituted by hydroxyl, heterocycyl, NR^{a}R^{b}, carboxy, and —C(O)—O—C₁₋₆alkyl); wherein R^{a} and R^{b} are each independently selected from hydrogen and C₁₋₆alkyl, or R^{a} and R^{b} together with the nitrogen to which they are attached may form a 4-7 membered heterocyclic ring;

and pharmaceutically acceptable salts, stereoisomers, esters and prodrugs thereof.

### EXAMPLES

The examples which follow are intended in no way to limit the scope of this disclosure but are provided to illustrate aspects of the present disclosure. Many other embodiments of this disclosure will be apparent to one skilled in the art.

### Example 1: Identification of MetAP-2 Inhibitors: Measuring Inhibition of HUVEC Cell Proliferation as a Function of Drug Exposure Time

Studies were conducted using HUVEC cells to investigate whether continuous exposure for >24 hours to MetAP-2 inhibitors may inhibit cell proliferation. Such inhibition of HUVEC proliferation may reflect an altered repair response to endothelial injury and promote clot initiation and propagation, e.g.,inhibiting proliferation and activation of other processes could affect the endothelium's ability to repair itself from injury and render it prothrombotic. Studies included HUVEC EC₅₀ concentrations used as a threshold for plasma exposures, and to determine the EC₅₀ values of two MetAP-2 inhibitors, beloranib and (3R,4S,5S,6R)-5-methoxy-4-((2R,3R)-2-methyl-3-(3-methylbut-2-en-1-yl)oxiran-2-yl)-1-oxaspiro[2.5]octan-6-yl 3-(2-morpholinoethyl)azetidine-1-carboxylate (Compound A) on the inhibition of HUVEC proliferation.

In vitro studies were conducted in cultured HUVECs (Lonza, Amboise, France). Cells were cultured in endothelial growth medium 2 cell culture media (EGM-2, Lonza, Amboise, France), passaged by seeding at 3×10³ cells/mL on T175 flasks, and allowed to reach approximately 80% confluence before experiments were conducted. Cells were trypsinized and counted using trypan blue exclusion method using an automated Cedex cell counter. HepG2 cells (American Type Culture Collection, Manassas, Va.) were seeded at 3×103 cells/mL in T150 flasks and grown to 60% confluence. Cells were exposed to compounds or beloranib at 10 nM final concentration for one hour then washed to remove compound, then cultured for different times until collection for MetAP2 analysis.

For LC-MS/MS experiments, cells were plated at 3×10⁴ cells/mL on tissue culture-treated T175 flasks at 35 mL/flask in triplicate and allowed to equilibrate overnight in a CO₂ incubator. On the day of the experiment, cell culture medium was removed, and fresh medium was added, along with drug or vehicle. Target compounds and beloranib were diluted in DMSO to yield stock concentrations of 10 μM. Subsequent dilutions were made in EGM-2 culture medium to yield a final DMSO concentration of 0.1% for cell incubations.

For LC-MS/MS sample preparation, cell culture media was removed, cells were washed with 10 mL of Dulbecco's phosphate-buffered saline (DPBS), and 500 μL of T-PER lysis buffer was added. Cell lysates were stored at −80° C. For assays measuring the cell cycle proteins p21 and p53, and the coagulation markers thrombomodulin (TM), plasminogen activator inhibitor-1 (PAI-1), and von Willebrand factor (vWF), cultured HUVECs were incubated with drug or vehicle for 4, 8, or 72 hours prior to compound washout and assessment of cell protein concentrations. For the 4- and 8-hour incubations, cells were maintained by standard culture for the remainder of the 72-hour incubation period. Cell cycle protein levels were assessed using enzyme-linked immunosorbent assays.

For analysis of drug binding to the MetAP2 enzyme active site, HUVEC lysates were denatured, reduced, and alkylated with IAM, then treated with chymotrypsin to generate the MetAP2 peptide (amino acids 312-320) EVEIDGKTY and either the Compound-bound MetAP2 peptide NNC^{[CAM]}AAH^{[Compound A]}Y or the beloranib-bound MetAP2 peptide NNC^{[CAM]}AAH^{[beloranib]}Y. Concentrations of the MetAP2-, compounds, or beloranib bound peptides were measured by LC-MS/MS. Measured peptide concentrations (in ng/mL) were converted to MetAP2-bound, compound-bound, and beloranib-bound protein concentrations (ng/mg total protein) in HUVEC lysates.

For analysis of MetAP2 enzymatic inhibitory activity of compounds in cells, the intact N-terminus of the MetAP2 substrate protein thioredoxin-1 (THX 1-6) was quantified. HUVEC lysates were denatured, reduced, and treated with endoproteinase Glu-C to generate the N-terminal methionine, THX 1-6 peptide, MVKQIE. Concentrations of MVKQIE were measured by LC-MS/MS. Measured peptide concentrations in ng/mL were converted to N-terminal methionylated THX 1-6 protein concentrations (ng/mg total protein) in HUVEC lysate.

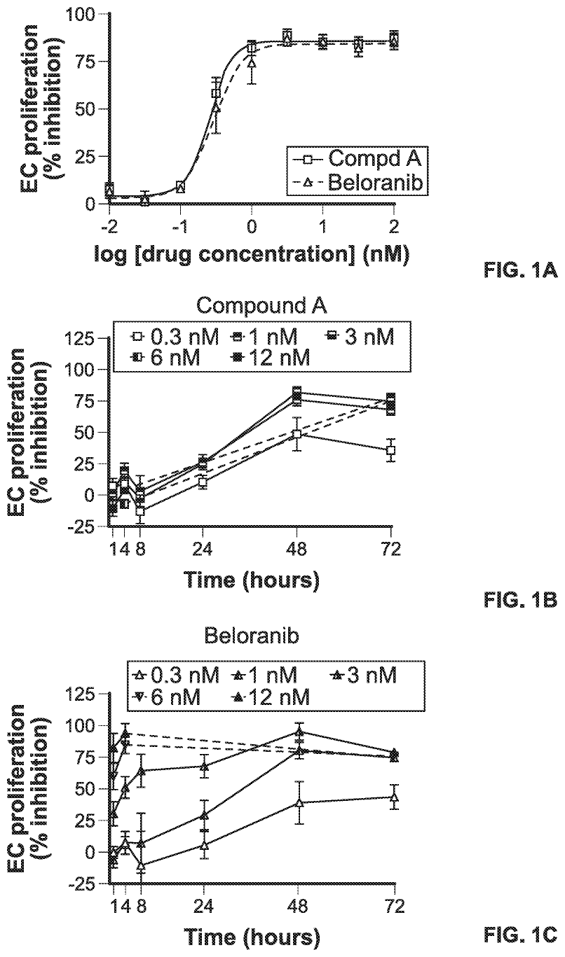

Compound A and beloranib decreased EC (endothetial cell) proliferation with continuous exposure over the 72-hour treatment period in a concentration-dependent manner (FIG. 1A). Both drugs were equally potent; the concentration that inhibited EC proliferation by 50% (EC₅₀) values were 0.24 nM and 0.27 nM for A and beloranib, respectively.

The effect of duration of exposure on inhibition of EC proliferation was then evaluated using shorter incubation times of 1, 4, 8, 24, and 48 hours. Exposure time and percent proliferation inhibition were examined by washing drug away after different intervals of time, e.g. 1 hr, 4 hr, 8 hr, 24 hr, 48 hr, and 72 hr and measuring proliferation. Robust inhibition of EC proliferation was observed with compound A only when exposure was continuous for 48 hours or longer (FIG. 1B). Short-term exposure (4 hours or less) of ECs to compound A at concentrations up to 50× the EC₅₀ had no effect on cell proliferation. In contrast, beloranib effects on EC proliferation occurred rapidly, with exposures as short as one hour, with effects starting as low as 11× the EC₅₀ (FIG. 1C).

For EC proliferation experiments, cells were plated at 0.3×105 cells/mL in a Corning 354649 96-well collagen-coated black microplate and allowed to equilibrate overnight in a CO₂ incubator. After incubation with drug or vehicle, EC proliferation was analyzed using a DNA dye (CyQUANT®) added to lysed cells to provide a fluorescent signal relative to the amount of DNA, allowing a measure of cell density.

Additional proliferation results with various fumagillin analog compounds are shown in Table 1.

| | | % | % | % | % |
|---|---|---|---|---|---|
| | | inhibition | inhibition | inhibition | inhibition |
| | HUVEC | 6XIC90 | 6XIC90 | 20XIC90 | 20XIC90 |
| Com- | Proliferation | 4 h | 24 h | 4 h | 24 h |
| pound | (EC50, nM) | exposure | exposure | exposure | exposure |
| Beloranib | 0.28 | 45 | 67 | 64 | 72 |
| Com- | 0.20 | 5.6 | 41 | 12.4 | 40 |
| pound A | | | | | |
| C | 0.54 | 25 | 62 | 16 | 62 |
| D | 0.31 | 0.18 | 29 | −0.4 | 33 |
| E | 0.20 | 14.6 | 48 | 12 | 53 |
| F | 0.37 | 4.6 | 36 | 0.4 | 43 |
| G | 0.13 | 1.4 | 30 | 7.3 | 31 |
| H | 0.08 | 9.4 | 35 | 7.4 | 39 |
| I | 0.36 | −0.1 | 32 | −4.3 | 31 |
| J | 0.78 | −3.9 | 33 | 2.2 | 47 |
| K | 0.59 | −4.5 | 35 | 1.9 | 51 |
| L | 0.30 | 14.3 | 45 | 16 | 56 |
| M | 0.42 | 1.8 | 34 | 8.8 | 46 |
| N | 0.22 | 5.5 | 46 | 8.6 | 49 |
| O | 0.17 | −1.2 | 41 | −5.9 | 40 |
| P | 0.29 | 13 | 47 | 9.7 | 54 |
| Q | 0.20 | 2.4 | 37 | 1.9 | 39 |
| R | 0.33 | 14 | 34 | 14 | 41 |
| S | 0.22 | 0.5 | 35 | 8.6 | 49 |
| T | 0.30 | 10 | 36 | 10 | 44 |
| U | 0.27 | −3.4 | 40 | 6.5 | 50 |

These studies show that a method for identifying MetAP-2 inhibitors, based on selecting those candidates compounds having minimum inhibition of HUVEC proliferation after short drug exposure times, provides a useful tool for identifying suitabled drug candidate compounds.

### Example 2: Identification of MetAP-2 Inhibitors: Measuring Molecular Markers of G1 Arrest and Altered Endothelial Coagulation Markers

A study was undertaken to assess changes in the levels of proteins associated with endothelial cell proliferation, as well changes in the levels of prothrombotic factors in endothelial cells, at discrete time intervals upon exposure to beloranib and Compound A and to investigate the extent of increased or decreased levels of molecular markers associated with disruptions in endothelial cell homeostatic balance, inhibition of endothelial cell proliferation, and G1 arrest—as well as activation of prothrombotic factors in endothelial cells in identifying MetAP-2 inhibitors having a lower risk of undesirable side effects (e.g., vein thrombosis).

HUVEC cells were exposed to compounds at concentrations of 1 nM, 3 nM, 10 nM, and 20 nM. After a duration of exposure at intervals of 4 hr, 8 hr, and 72 hr, the cells were lysed and the prepared lysates were stored at −80 ° C. until protein levels were assayed and measured in the cell lysate. The protein assays performed included measurements of total p21, thrombomodulin, total p53, PAI-1, and von Willibrand factor (vWF). All values obtained were normalized against protein concentration.

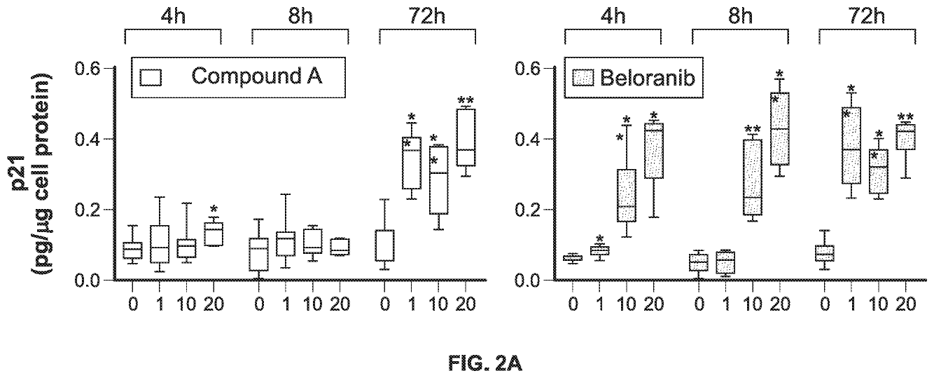

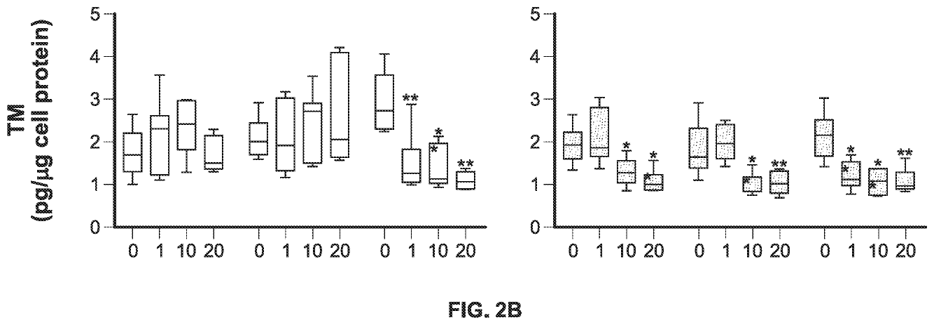

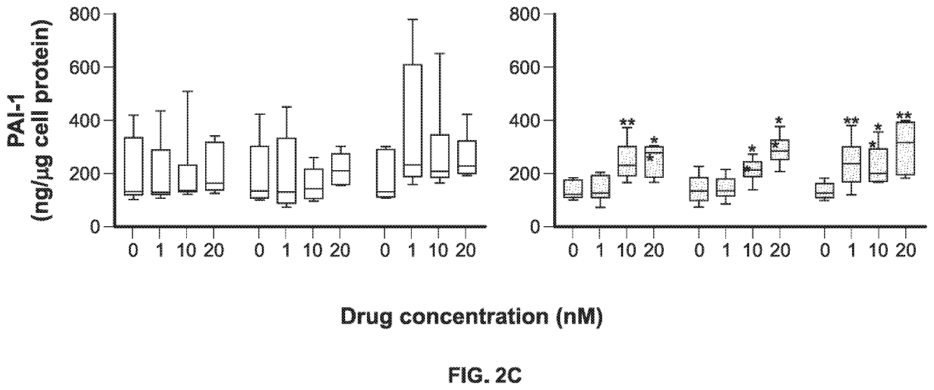

All concentrations of Compound A increased p21 (FIG. 2A, left) and reduced TM protein levels (FIG. 2B, left) after 72 hours of incubation, but there were no meaningful changes with shorter incubations of 4 or 8 hours. Beloranib also increased p21 (FIG. 2A, right) and reduced TM levels (FIG. 2B, right) after 72 hours of incubation at all concentrations. In contrast, short 4- or 8-hour incubations with the higher concentrations of beloranib (10 and 20 nM) increased p21 and reduced TM protein levels. Compound A had no effect on PAI-1 protein levels at any of the concentrations or time points (FIG. 2C, left), whereas beloranib increased PAI-1 levels at all concentrations at 72 hours and at 10 and 20 nM at the earliest timepoint (FIG. 2C, right).

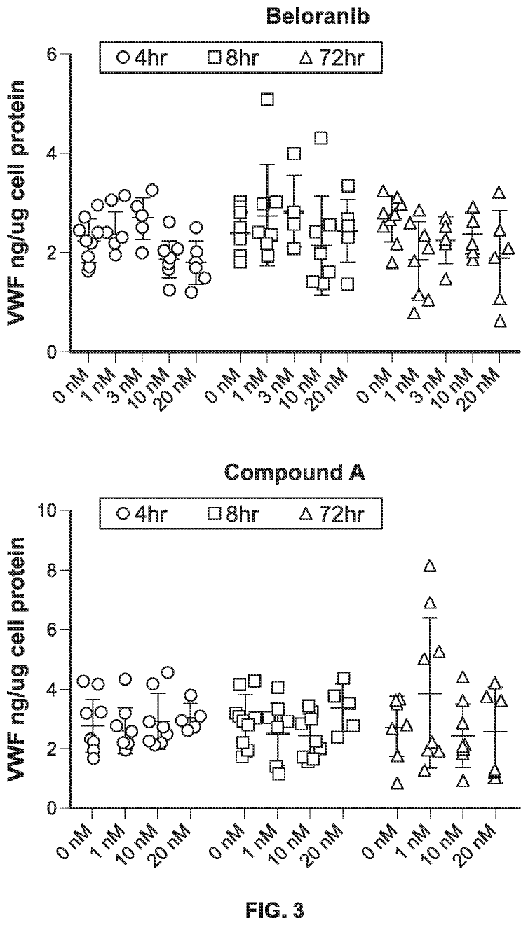

FIG. 3 shows measurements of vWF levels after exposure to compounds at discrete time intervals and concentrations. As shown in FIG. 3, no effects on vWF levels were observed for either beloranib or Compound A.

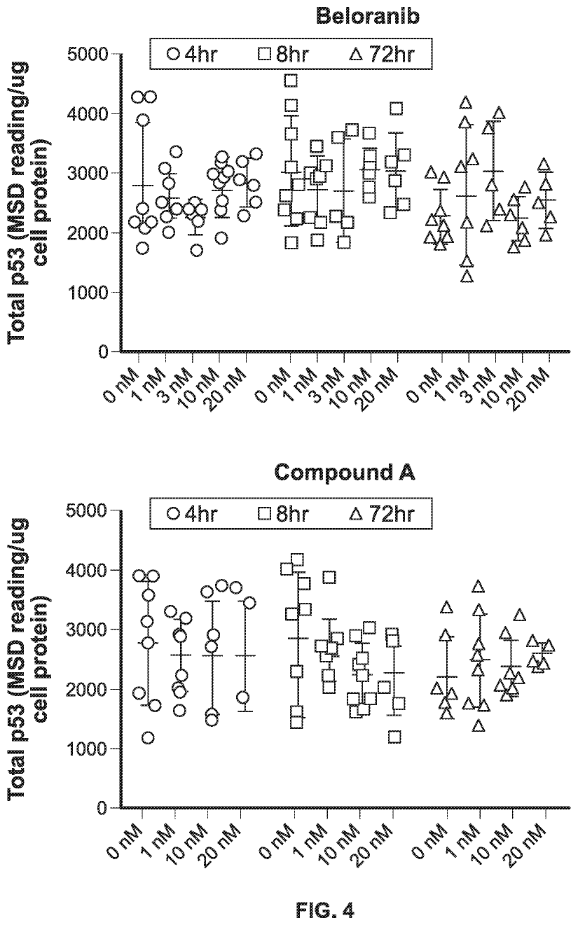

FIG. 4 shows measurements of p53 protein levels after exposure to compounds at discrete time intervals and concentrations. As shown in FIG. 4, no effects on p53 protein levels were observed for either beloranib or Compound A.

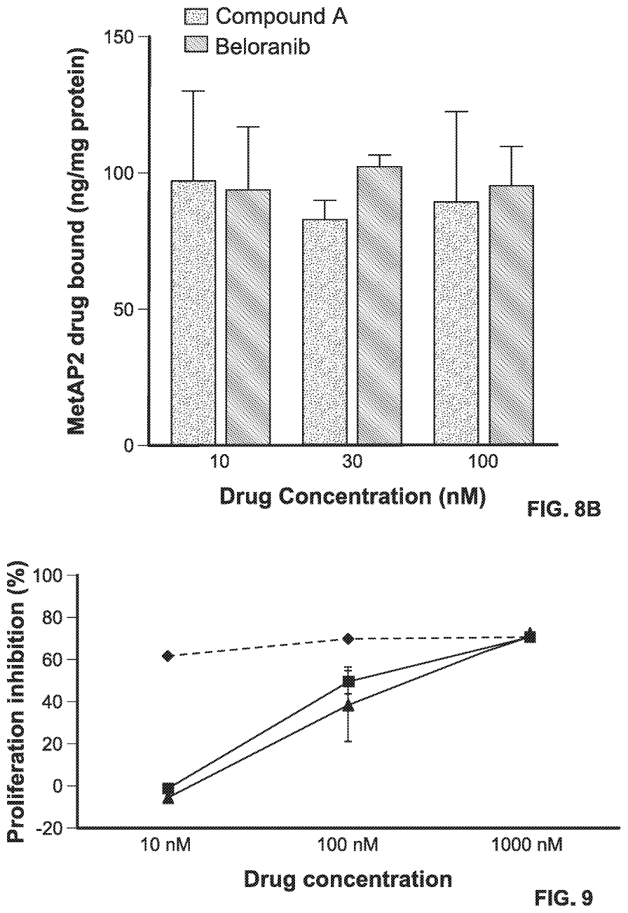

The effects of compound A and beloranib on MetAP2 target engagement was also conducted in HUVECs. Incubation of HUVECs with 10, 30, or 100 nM of Compound A or beloranib for 1 hour followed by washing away of compound from the culture media resulted in a more than 10-fold higher cell-associated concentration of beloranib when compared to cell-associated levels of Compound A at any given incubation concentration (FIG. 8A). Despite the differences in cell-associated drug levels, there were similar amounts of drug bound covalently to the MetAP2 active site across the concentration range for both Compound A and beloranib (FIG. 8B). Thus, similar and maximal binding to MetAP2 protein occurred with the 10 nM concentration of both Compound A and beloranib and subsequent MetAP2 target engagement studies employed a 10 nM concentration of both drugs.

A washout assay protocol is as follows: Day 1: plate cells: Plate cells at 0.3×10⁴ cells/well in collagen coated 96-well plates; Incubate cells at 37° C., 5% CO₂ in a humidified atmosphere overnight. Day 2: dilute and add MetAP2 inhibitor compounds: Dilute compounds in cell culture medium to the appropriate concentration. Add medium containing compound onto cells. Incubate cells at 37° C., 5% CO₂ in a humidified atmosphere for the appropriate timepoint. Washout: After the indicated time, remove medium from the relevant samples and wash twice with PBS. Replace with fresh medium Incubate at 37° C., 5% CO₂ in a humidified atmosphere until final incubation time. Day 5-8 (72-140 h): Cell proliferation measurement: At the indicated time remove medium from cells and wash twice with PBS. Measure cell proliferation using CyQuant assay.

An exemplary additional result is shown in FIG. 9. 3×10³ HUVECs were plated in collagen coated 96-well plates and treated with beloranib, and two candidate compounds for 1 h. Compound was washed out at the indicated time and replaced with fresh medium. The cells were incubated for 72 hrs and cell proliferation was measured to assess inhibition. The results show that 10 nM of the two compounds (solid lines) do not inhibit cell proliferation after 1 hour of exposure, while ˜40% inhibition is reached with 100 nM, and ˜60% with 1000 nM drug. Beloranib (dashed line) was inhibitory at all concentrations. Error bars represent the standard error of the mean for each condition (n=8 replicates per test condition pooled from 3 experiments).

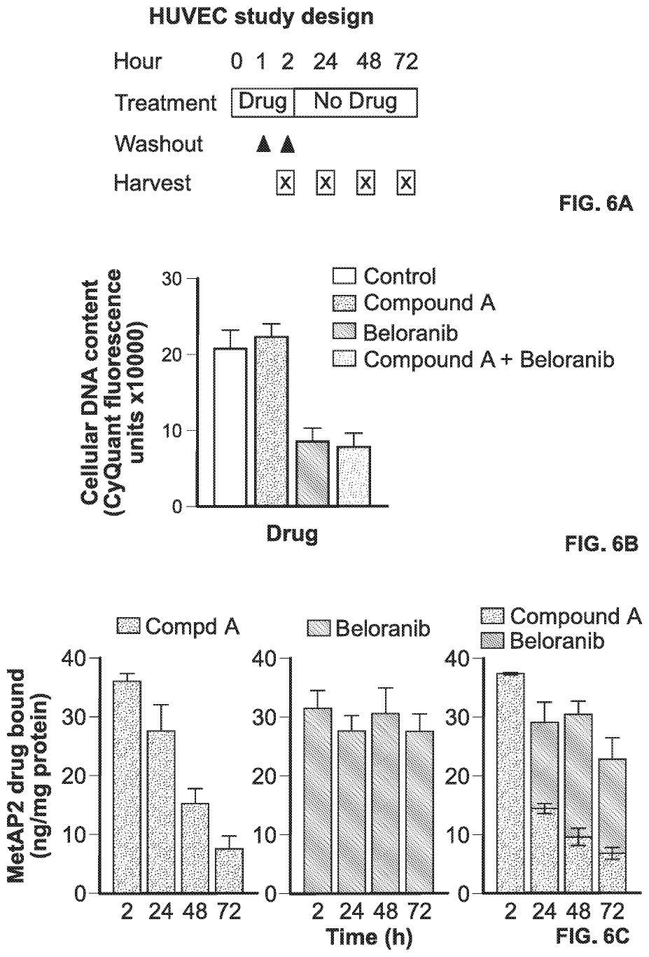

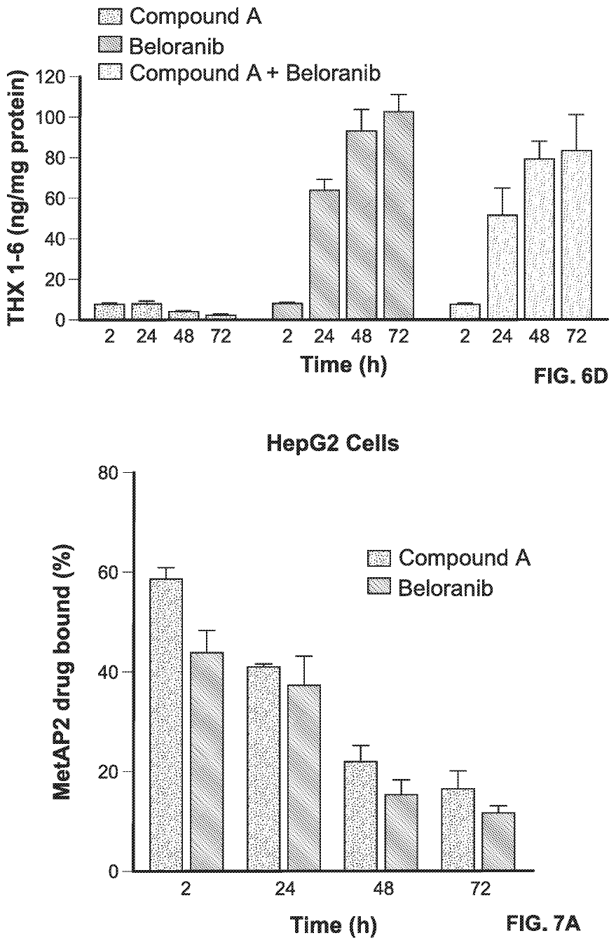

A second experiment was conducted in ECs to address if preincubation of the MetAP2 enzyme with a saturating concentration of Compound A (10 nM) would prevent subsequent binding by beloranib (10 nM) and influence cell proliferation, MetAP2 target engagement, or MetAP2 enzymatic activity. HUVECs were evaluated at various times over a 72-hour period after a 2-hour incubation with either Compound A alone, beloranib alone, or a sequential 1-hour incubation with Compound A followed by a 1-hour incubation with beloranib (FIG. 6A). Compound A alone had no effect on cell proliferation when measured at 72 hours, whereas beloranib alone reduced cell proliferation by approximately 60% (FIG. 6B). When HUVECs were first exposed to Compound A for one hour, then drug was washed out, then subsequently exposed to beloranib for one hour, and again drug was washed out, the result was an inhibition of cell proliferation (FIG. 6B), indicating that preincubation with Compound A does not prevent subsequent inhibition of cell proliferation by beloranib.

MetAP2 target engagement was assessed by measuring the concentration of MetAP2 protein with drug covalently bound to the active site His-231 residue (FIG. 6C). In cells that were only exposed to compound A for 2 hours, target engagement decreased over the 72-hour time period. In contrast, in cells only exposed to beloranib for 2 hours, target engagement was persistent across the 72-hour time period. Following sequential exposures of compound A for one hour, then beloranib for one hour, only Compound A initially occupied the MetAP2 enzyme active site. With continued culture, Compound A in the enzyme active site was gradually replaced by beloranib over the 72-hour period, despite both compounds being washed after the initial 2-hour incubation.

Target engagement of the MetAP2 enzyme can also be measured functionally through the accumulation of the MetAP2 substrate thioredoxin that retains its intact N-terminal methionine residue (THX 1-6). FIG. 6D shows that the THX 1-6 concentrations were consistently low over the 72-hour culture following exposure to only Compound A for 2 hours. In contrast, a 2-hour exposure to beloranib resulted in a marked increase of THX 1-6 over the 72 hours. In the case of sequential exposure of compound A (1 hour) followed by beloranib (1 hour) the levels of THX 1-6 were also markedly increased over the 72 hours, indicative of sustained target engagement in beloranib-exposed EC cells despite no ongoing extracellular exposure to the drug.

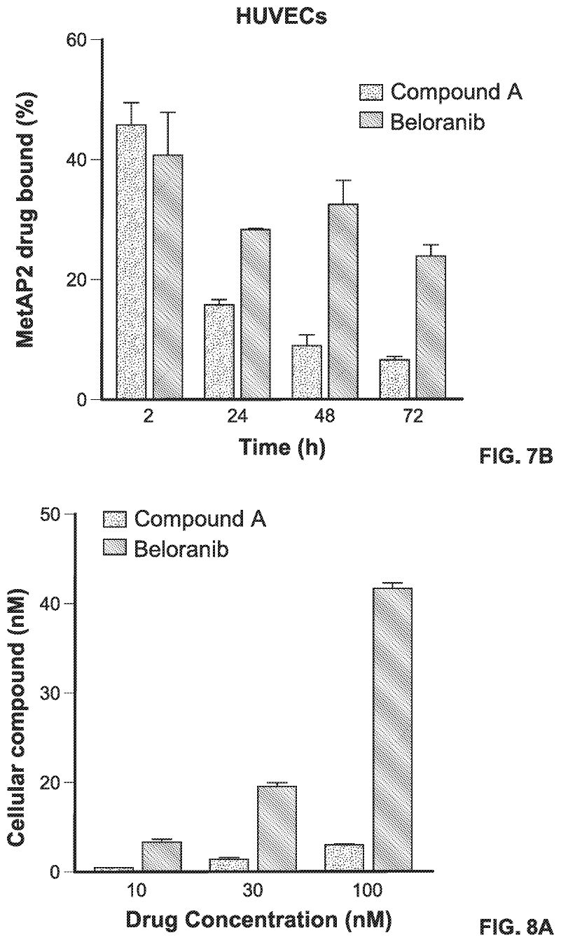

Target engagement kinetics were also assessed in HepG2 cells. For this assessment, HepG2 cells were exposed to either Compound A or beloranib for 2 hours. Drug was then washed out and levels of MetAP2 with drug bound in the active site were measured. FIG. 7A shows that both Compound A and beloranib are found in the active site of MetAP2 after the 2-hour exposure. Upon further culture, the proportion of MetAP2 that contained drug in the active site declined similarly over the 72 hours for both Compound A and beloranib. In comparison, HUVECs exposed to the drug in a similar manner showed a sustained level of beloranib in the MetAP2 active site over 72-hours culture, whereas Compound A target engagement declined (FIG. 7B).

Methods of identifying MetAP-2 inhibitors comprising measuring vimentin protein levels are conducted according to procedures analogous to the above. Methods of identifying MetAP-2 inhibitors comprising exposing a cell to a potential MetAP-2 compound in a culture medium; retrieving a sample from the cell and/or culture medium at one or more predetermined time points; analyzing the sample for increased or decreased expression levels of at least one gene each selected from the group consisting of p53, p21, eNOS, PAI-1, TM, RF, KLF2, MDM2, and vimentin; and identifying the compound as suitable for treatment of obesity based on the increased expression level or decreased expression level are conducted according to procedures analogous to the above and known by a skilled person in the art.

### Example 3 Effect on Coagulation Markers in Dogs

Male beagle dogs 10 to 20 months of age (Marshall BioResources, North Rose, NY) were individually housed at the testing facility. Dogs (n=6/group) received 2 mg/kg Compound A s.c. Q3D on Days 1, 4, 7, and 10. In a separate study, dogs (n=6/group) received 0.6 mg/kg beloranib s.c. Q3D for a total of 8 planned doses (Days 1, 4, 7, 10, 13, 16, 19, and 22). Animals were observed twice daily for signs of ill health, morbidity, mortality, injury, and viability; and daily detailed clinical observations were conducted (including urine/fecal examination and hands-on examination). Body weight and food intake were recorded daily. Blood was collected before each dose and 4 hours after each dose for hematology (e.g., platelet count), coagulation parameters (e.g., thrombin time, antithrombin III), and protein panel analysis (e.g., D-dimer) (Nextcea, Woburn, Mass.).

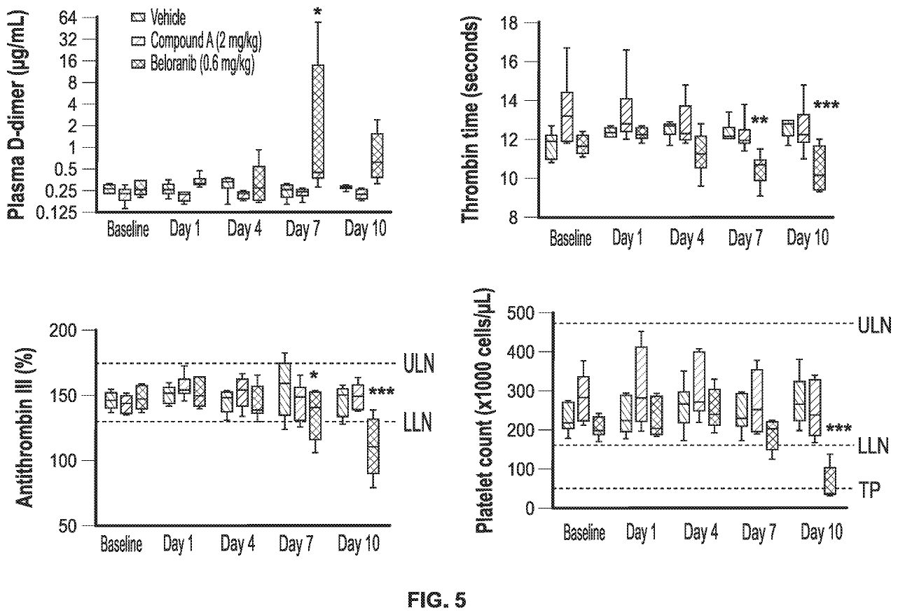

In studies of beloranib toxicology in dogs, some animals developed evidence of impaired hemostasis (i.e., marked reductions in platelet count, bleeding gums, bloody stool) within 2 weeks of drug administration at doses of 0.6 mg/kg s.c. Q3D. This altered hemostasis led to a focused investigation of the effects of compound A and beloranib on hematology and coagulation parameters in dogs over the first 10 days of treatment. Compound A (2 mg/kg s.c. Q3D) administered to dogs for 10 days was well tolerated and produced no adverse changes in clinical observations, hematology (e.g., platelet count), or the coagulation markers, D-dimer, thrombin time, and antithrombin III (FIG. 5). Weight loss was observed over the course of the study, but food intake was unchanged, and all animals survived through the scheduled dosing period. In a separate study, beloranib (0.6 mg/kg s.c. Q3D) administration to dogs was accompanied by increased D-dimer concentrations and decreased thrombin time, antithrombin III, and platelet count (FIG. 5). At 0.6 mg/kg, beloranib was not well tolerated with fecal changes, lethargy, emesis, red and/or black feces, and decreased activity.

### INCORPORATION BY REFERENCE

All publications and patents mentioned herein are hereby incorporated by reference in their entirety for all purposes as if each individual publication or patent was specifically and individually incorporated by reference. In case of conflict, the present application, including any definitions herein, will control.

### EQUIVALENTS

While specific embodiments of the subject invention have been discussed, the above specification is illustrative and not restrictive. Many variations of the present disclosure will become apparent to those skilled in the art upon review of this specification. The full scope of the disclosure should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

What is claimed is:

1. A method for identifying MetAP-2 inhibitors suitable for human treatment of disorders, comprising:
exposing test cells or test tissue to a MetAP-2 inhibitor candidate compound;
measuring the inhibition of proliferation in the test cells or test tissue in discrete time intervals; and
selecting the candidate compound as suitable for treatment by identifying whether the candidate compound shows minimal inhibition of proliferation at a designated time of exposure to the test cells or test tissue.

2. The method of claim 1, wherein the test cells are venous endothelial cells.

3. The method of claim 1 or 2, wherein the test cells are human umbilical vein endothelial cells (HUVEC).

4. The method of any one of claims 1-3, wherein the exposing step comprises exposing the cells to a MetAP-2 inhibitor candidate compound at a concentration of about 0.15 nM to about 5 nM or more.

5. The method of any one of claims 1-4, wherein the exposing step comprises exposing the cells to a MetAP-2 inhibitor candidate compound at a concentration of about 10 to 100 times or more the IC₅₀ against MetAP-2 of the candidate compound in the same type of test cell.

6. The method of any one of claims 1-4, wherein the exposing step comprises exposing the cells to a MetAP-2 inhibitor candidate compound at a concentration of about 1 to about 50 times or more the IC₉₀ against MetAP-2 of the candidate compound in the same type of test cell.

7. The method of claim 5 or 6, wherein the concentration is about 6 to about 30 times the IC₅₀ or IC₉₀.

8. The method of any one of claims 5-7, wherein the concentration is about 20 times the IC₉₀.

9. The method of any one of claims 1-4, wherein the exposing step comprises exposing the cells to a MetAP-2 inhibitor candidate compound at a concentration of about 1 to about 50 times the EC₅₀ of the candidate compound exposed to HUVEC cells for 72 hours.

10. The method of any one of claims 1-4, wherein the exposing step comprises exposing the cells to a MetAP-2 inhibitor candidate compound at a concentration of about 5 to about 25 times or more the EC₉₀ of the candidate compound when exposed to HUVEC cells for 72 hours.

11. The method of any one of claims 1-4, wherein the exposing step comprises exposing the cells to a MetAP-2 inhibitor candidate compound at a concentration of about 5 to about 25 times or more the EC₅₀ of the candidate compound when exposed to HUVEC cells for 72 hours.

12. The method of any one of claims 1-11, wherein the designated time of exposure is 4 hours.

13. The method of claim 11, wherein the minimal inhibition of cell proliferation at 4 hours is less than 50%.

14. The method of claim 11, wherein the minimal inhibition of cell proliferation at 4 hours is less than 25%.

15. The method of any one of claims 1-11, wherein the minimal inhibition of cell proliferation at 4 hours is less than or about 15%, or less than or about 10%, or less than or about 5%.

16. The method of any one of claims 1-11, wherein the designated time of exposure is 24 hours.

17. The method of any one of claim 16, wherein the minimal inhibition of cell proliferation at 24 hours is less than 40%.

18. The method of claim 16, wherein the minimal inhibition of cell proliferation at 24 hours is less than 25%.

19. The method of claim 16, wherein the minimal inhibition of cell proliferation at 24 hours is less than or about 15%, or less than or about 10%.

20. The method of any one of claims 1-19, further comprising measuring p21 cell protein.

21. The method of claim 20, wherein selecting the candidate compound further comprises identifying whether the candidate compound increases p21 protein concentration more than about 4 fold at 72 hours exposure to the venous endothelial cells.

22. The method of any one of claim 20 or 21 wherein selecting the candidate compound further comprises identifying whether the candidate compound significantly increases p21 protein at a MetAP-2 inhibitor concentration of 10 nM or 20 nM at short exposure time.

23. The method of claim 22, wherein the short exposure time is 4 or 8 hours.

24. The method of any one of claims 1-23, further comprising measuring thrombomodulin concentration.

25. The method of any one of claims 1-24, further comprising measuring PAI-1 cell protein concentration.

26. The method of any one of claims 1-25, further comprising measuring one or more of vWF, p53, D-Dimer, and vimentin protein.

27. A method for identifying MetAP-2 inhibitors suitable for human treatment of disorders, comprising:
providing a concentration parameter of MetAP-2 selected from the group consisting of IC₅₀, IC₉₀, EC₅₀ as measured at 72 hours in a HUVEC cell, and EC₉₀ as measured at 72 hours in a HUVEC cell;
exposing HUVEC to a MetAP-2 inhibitor candidate compound at a concentration of about 5 to about 40 times the concentration parameter;
measuring the inhibition of proliferation in the test cells at 4 hours or at 24 hours; and
selecting the candidate compound as suitable for treatment by identifying whether the candidate compound shows less than or about 15% inhibition of cell proliferation at about 4 hours of exposure to the HUVEC cells.

28. A method for identifying a candidate MetAP-2 inhibitor compound suitable for human treatment of a disorder, comprising:
exposing a cell to a potential MetAP-2 compound in a culture medium;
retrieving a sample from the cell and/or culture medium, at one or more predetermined time points;
analyzing the sample for increased or decreased expression levels of at least one gene each selected from p53, p21, eNOS, PAI-1, TM, RF, KLF2, MDM2, and vimentin; and
identifying the compound as suitable for treatment to a human patient based on the increased expression level or decreased expression level.

29. The method of claim 28, wherein the cell is a venous endothelial cell.

30. The method of claim 28 or 29, wherein the cell is a HUVEC cell.

31. A method for identifying a candidate MetAP-2 inhibitor compound suitable for treatment of a disorder in a human patient, comprising:
exposing a cell to a potential compound in a culture medium;
retrieving a sample from the cell and/or culture medium, at one or more predetermined time points;
analyzing the sample for increased or decreased levels of PAI-1 cell protein; and
identifying the compound as suitable for treatment of a disorder based on the increased or decreased PAI-1 cell protein levels.

32. A method for identifying MetAP-2 inhibitors having minimal persistant cell proliferation and therefore suitable for human treatment of disorders, comprising:
exposing test cells or tissue to a MetAP-2 inhibitor candidate compound for a first incubation time;
performing a washout of the candidate compound from the test cells or tissue after the first incubation time;
continuing incubation of the cells in the absence of the candidate compound for a second incubation time;
measuring the inhibition of proliferation in the test cells or tissue in discrete time intervals; and
selecting the candidate compound as suitable for treatment by identifying whether the candidate compound shows minimal inhibition of cell proliferation at a designated time after the washout to the cells or tissue.

33. A method for identifying a candidate MetAP-2 inhibitor compound suitable for treatment of a disorder in a human patient, comprising:
exposing a cell or tissue to a potential MetAP-2 inhibitor in a culture medium;
retrieving a sample from the cell and/or culture medium, at one or more predetermined time points;
optionally performing a washout of the candidate compound from the test cells or tissue after a first incubation time and continuing incubation;
analyzing the sample for for increase or decreased gene expression levels and/or increased or decreased protein levels; and
identifying the compound as suitable for treatment of a disorder based on the increased expression level or decreased protein level.

34. The method of any one of claims claim 31-33, wherein the cell is a HUVEC cell.

35. The method of any one of claims 1-34, wherein the MetAP-2 candidate compound has an IC₅₀ against MetAP-2 of about 0.1 nM to about 5 nM.

36. The method of any one of claims 1-35, wherein the method further comprises assessing efficacy of the candidate compound for the disorder in a cell, tissue, organ or animal.

37. The method of any one of claims 1-36, wherein the disorder is a metabolic disease.

38. The method of any one of claims 1-36, wherein the disorder is obesity and/or a co-morbidity thereof.

39. The method of any one of claims 1-36, wherein the disorder is type 2 diabetes or latent autoimmune diabetes.

40. The method of any one of claims 1-36, wherein the disorder is chronic inflammatory disease or impaired wound healing.

41. The method of any one of claims 1-36, wherein the disorder is an inflammatory disease.

42. The method of claim 41, wherein the inflammatory disease is selected from the group consisting of inflammatory bowel disease, Kawasaki disease, Sjogren's syndrome, systemic lupus erythematosus, rheumatoid arthritis, psoriatic arthritis, chronic obstructive pulmonary disease, and psoriasis.
